(19) United States
(12) Patent Application Publication
(10) Pub. No.: US 2022/0104790 A1
(43) Pub. Date: Apr. 7, 2022

(54) CONTINUOUS AND DYNAMIC EJECTION FRACTION DETERMINATION

(71) Applicant: Caption Health, Inc., Brisbane, CA (US)

(72) Inventors: Charles Cadieu, Burlingame, CA (US); Ha Hong, Pleasant Hill, CA (US); Kilian Koepsell, San Francisco, CA (US); Ali Chaudhry, San Francisco, CA (US); Nicolas Poilvert, Seattle, WA (US); Michael G. Cannon, Haverford, PA (US); Nripesh Parajuli, San Mateo, CA (US)

(21) Appl. No.: 17/061,578

(22) Filed: Oct. 2, 2020

(51) Int. Cl. A61B 8/06; A61B 8/08; A61B 8/00; G06T 5/50

(52) U.S. Cl. CPC A61B 8/065; A61B 8/5207; A61B 8/461; G06T 5/50; G06T 2207/10132; G06T 2207/20084; G06T 2207/30048; G06T 2207/20216; G06T 2207/30168; G06T 2207/20081

(57) ABSTRACT

The continuous and dynamic computation of ejection fraction (EF) includes training a neural network with different sets of cardiac imaging data acquired of a ventricle for different hearts and a known EF for each of the sets and then loading the trained neural network into memory of a computer. Afterwards, contemporaneous sets of imaging data of a ventricle of a heart are continuously acquired according to a specified view. For each corresponding set of imaging data, an image quality value may then be computed, and the corresponding set of imaging data may be provided to the neural network. The neural network, in response, provides, as output, an EF determination output without tracing a ventricle boundary of the heart. Thereafter, both the computed image quality value and the EF determination output may be displayed in a display of the computer.

### BACKGROUND OF THE INVENTION

### Field of the Invention

The present invention relates to the systolic function of the heart and more particularly ejection fraction measurement.

### Description of the Related Art

The ejection fraction is a measurement of the systolic function of the heart that refers to the percentage of blood leaving the heart at each contraction. Specifically, during each pumping cycle of the heart, the heart both contracts and also relaxes. When the heart contracts, the heart ejects blood from its two pumping chambers, known as the left ventricle and the right ventricle. Conversely, when the heart relaxes, both ventricles refill with blood. Of note, no matter how forceful the contraction of the heart, the heart does not pump all of the blood out of each ventricle. Instead, some blood remains. Hence, the term “ejection fraction” refers to the percentage of blood that is pumped out of a filled ventricle with each heartbeat.

Of the two ventricles, the left ventricle is the pumping chamber of the heart that pumps oxygenated blood through the ascending aorta to the rest of the body, and the right ventricle is the chamber that pumps blood to the lungs for oxygenation. The ejection fraction of the left or right ventricle may be measured through the use of several different imaging techniques. The most common technique is the echocardiogram in which the ejection fraction is measured by sound-wave produced images of the heart and the blood pumping through the heart. Other alternatives to echocardiography include the use of magnetic resonance imaging (MRI), computerized tomography (CT) and nuclear medicine scanning, and catheter-based imaging.

Echocardiograms may be performed with two or three dimensional imaging modes. Echocardiograms are performed using multiple access windows to the heart, such as the parasternal, apical, or subcostal access windows. They may also be performed with endoscope-based transesophageal transducers placed in the esophagus or stomach. The echocardiogram access window allows multiple separate views that are formed by angling or rotating the transducer. When an echocardiogram study is generated using multiple windows and multiple views, diagnostic accuracy may be increased.

Current ejection fraction measurement methods tend to inaccurately assess disease conditions of the heart. This error can lead to the delayed treatment of patients, and the significant worsening of disease conditions during the delay. In this regard, for example, two-dimensional echocardiography, the most common method, relies upon Simpson's Biplane methodology to produce a measurement. In particular, in the Simpson's Biplane methodology, the end systolic and end diastolic volumes of the ventricle are measured so as to compute a fractional difference. But, in doing so, the ventricle border needs to be manually traced by a human reader, or automatically traced using endocardial border detection methods, both of which methods are subject to error. Then, the ventricle volume is assumed to be composed of a finite number, usually twenty, of elliptical cylinders, which while convenient, is not accurate. Moreover, this methodology relies on finding the exact end systolic and end diastolic image frames, often nontrivial step that can introduce errors if not done accurately. Three dimensional echocardiograms may rely less on assumptions about the shape of the ventricle for calculating ejection fraction than two-dimensional mode, but it still requires the manual or automated and error-prone detection of endocardial borders. As a result, current methods in ejection fraction measurement perform measurements in a way that is neither optimized nor reproducible despite the advances in modern diagnostic technologies.

### BRIEF SUMMARY OF THE INVENTION

Embodiments of the present invention address deficiencies of the art in respect to the determination of ejection fraction and provide a novel and non-obvious method, system and computer program product for the continuous and dynamic automated computation of ejection fraction. In an embodiment of the invention, a method for continuously and dynamically computing ejection fraction includes first training a neural network with different sets of cardiac imaging data acquired of a ventricle for different hearts and a known ejection fraction for each of the sets and then loading the trained neural network into memory of a computer. Afterwards, contemporaneous sets of imaging data of a ventricle of a heart may be continuously acquired according to a specified view. For each corresponding one of the sets of imaging data, an image quality value may then be computed for the corresponding one of the sets, and the corresponding one of the sets of imaging data may be provided to the neural network. The neural network, in response, provides, as output, an ejection fraction determination output without tracing a ventricle boundary of the heart. Thereafter, both the computed image quality value and the ejection fraction determination output may be displayed in a display of the computer.

In one aspect of the embodiment, the method additionally includes ending the continuous acquisition of the contemporaneous sets of imaging data in response to the computation of a quality value that exceeds a threshold value. In another aspect of the embodiment, a different view may be selected with the continuous acquisition of the contemporaneous sets of imaging data utilizing the different view. The selection of the different view may occur at the direction of an operator, or the selection of the different view may occur in consequence of the determination that a threshold number of sets of imaging data have been acquired without computing a quality value that exceeds the threshold value.

In yet another aspect of the embodiment, the continuous acquisition of the contemporaneous sets of imaging data can include an averaging at least two successive ones of the sets of imaging data and so as to provide the averaged successive sets of imaging data to the neural network. Similarly, the method can include an averaging of an ejection fraction determination for multiple different ones of the contemporaneous sets of imaging data so as to produce an averaged ejection fraction determination for display in the computer. In the latter instance, each ejection fraction determination included in the averaging can be weighted according to a corresponding computed image quality value. Alternatively, each ejection fraction determination included in the averaging may be weighted according to a corresponding image view used in acquiring the imaging data from which the ejection fraction had been determined.

In another embodiment of the invention, a data processing system can be adapted for continuously and dynamically computing ejection fraction. The system includes a host computing platform having one or more computers, each with memory and at least one processor. The system also includes a display communicatively coupled to the host computing platform and a data store storing therein a neural network trained with different sets of cardiac imaging data acquired of a ventricle for different hearts and a known ejection fraction for each of the sets. Finally, the system includes an ejection fraction determination module.

The module includes computer program instructions enabled while executing in the host computing platform to load the trained neural network into the memory of the computer and to continuously acquire contemporaneous sets of imaging data of a ventricle of a heart according to a specified view. As such, the program instructions then, for each corresponding one of the sets of imaging data, compute an image quality value for the corresponding one of the sets, provide the corresponding one of the sets of imaging data to the neural network and receive as output from the neural network, an ejection fraction determination output without tracing a ventricle boundary of the heart. Finally, the program instructions, while executing in the host computing platform, display both the computed image quality value and the ejection fraction determination output in the display.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for continuously and dynamically computing ejection fraction;

FIG. 2 is a schematic illustration of a data processing system adapted for the continuous and dynamic computation of ejection fraction; and,

FIG. 3 is a flow chart illustrating a process for continuously and dynamically computing ejection fraction.

### DETAILED DESCRIPTION OF THE INVENTION

Embodiments of the invention provide for the continuous and dynamic computation of ejection fraction in a data processing system. In accordance with an embodiment of the invention, utilizing one or more views of a heart, different sets of contemporaneous imagery of the heart or a portion thereof are acquired ultrasonically and submitted to a neural network trained to correlate different sets of imagery of the heart with different ejection fraction values without requiring a tracing of the boundary of any portion of the heart. The neural network then produces a corresponding ejection fraction value for each submitted set of imagery which may then be displayed in a display of a computer. In this way, rather than receiving merely a finalized value for ejection fraction from a single set of acquired imagery of the heart, a continuously display of ejection fraction determinations may be provided in real-time as the heart is subjected to ultrasound imaging such that an operator may assess the stability of the ejection fraction determination during the course of the ultrasound imaging.

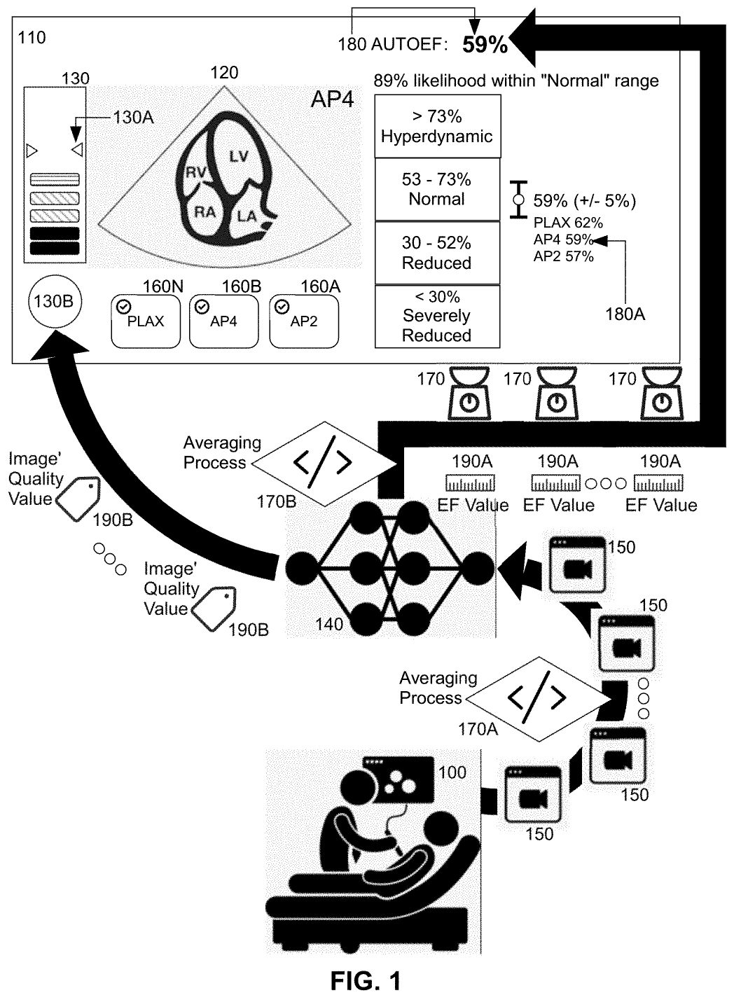

In further illustration, FIG. 1 is a pictorial illustration of a process for continuously and dynamically computing ejection fraction. As shown in FIG. 1, an ultrasound diagnostic imaging device 100 acquires a continuous stream of video clips 150 in respect to a selected one of multiple different views 160A, 160B, 160N of a heart. Each of the video clips 150, either in real time, or after having been placed in persistent storage, are provided to a neural network 140 trained both to produce for each received one of the video clips 150 a correlated ejection fraction value 190A and also to produce a quality value 190B for each corresponding one of the video clips 150. As the neural network 140 produces an ejection fraction value 190A, the ejection fraction value is displayed as a contemporaneous ejection fraction value 180. In the event that the video clips 150 have been acquired in real time, the ejection fraction value is displayed as a contemporaneous ejection fraction value 180 within a user interface 110 to the ultrasound diagnostic imaging device 100. Otherwise, the ejection fraction value may be displayed separate and apart from the user interface 110 to the ultrasound diagnostic imaging device 100. In any event, as to the either circumstance, a summary indicator 180A also may be presented showing an ejection fraction received from the neural network 140 for each selected one of the views 160A, 160B, 160N.

Concurrently, as the neural network 140 produces a quality value 190B for a corresponding one of the video clips 150 from which the ejection fraction value 190A had been derived, the quality value 190B is displayed in a quality meter 130 in the user interface 110 to the ultrasound diagnostic imaging device 100. The process can continue for each ejection fraction value 190A received from the neural network 140 for each corresponding video clip 150 acquired by the ultrasound diagnostic imaging device 100. In one aspect of the embodiment, a threshold quality marker 130A can be included in the quality meter 130 so as to indicate when the quality value 190B received from the neural network 140 exceeds a threshold value.

In response to a received one of the quality values 190B exceeding the threshold value, a success icon 130B can be activated to indicate that the most recently acquired one of the video clips 150 is of sufficient quality to produce an accurate corresponding one of the ejection fraction values 190A. Conversely, in response to a threshold period of time without having acquired one of the video clips 150 of sufficient quality, the selected one of the views 160A, 160B, 160N may change automatically, or a prompt may be rendered in the user interface 100 recommending a selection of a different one of the views 160A, 160B, 160N. Likewise, in response to a threshold number of acquired ones of the video clips 150 of quality determined by the neural network 140 to be below the threshold value, the selected one of the views 160A, 160B, 160N may change automatically, or a prompt may be rendered in the user interface 100 recommending a selection of a different one of the views 160A, 160B, 160N.

Optionally, an averaging process 170A may be applied to two or more successive acquired ones of the video clips 150 so that on average of two or more successive ones of the video clips 150 are presented to the neural network for correlation into a resultant ejection fraction value 190A. As another option, an averaging process 170B may be applied to two or more successive ones of the resultant ejection fraction values 190A so as to provide for display as the contemporaneous ejection fraction value 180, an average value of the two or more successive ones of the resultant ejection fraction values 190A. As part of the averaging process 170B, different weights 170 may be applied to respectively different ones of the two or more successive ones of the resultant ejection fraction values 190A dependent upon which of the views 160A, 160B, 160N had been utilized in acquiring the corresponding video clips 150. Consequently, the actions of the operator in acquiring the corresponding video clips 150 can be smoothed when visualizing the relationship between image quality of an acquired one of the video clips 150 and the contemporaneous ejection fraction value 180.

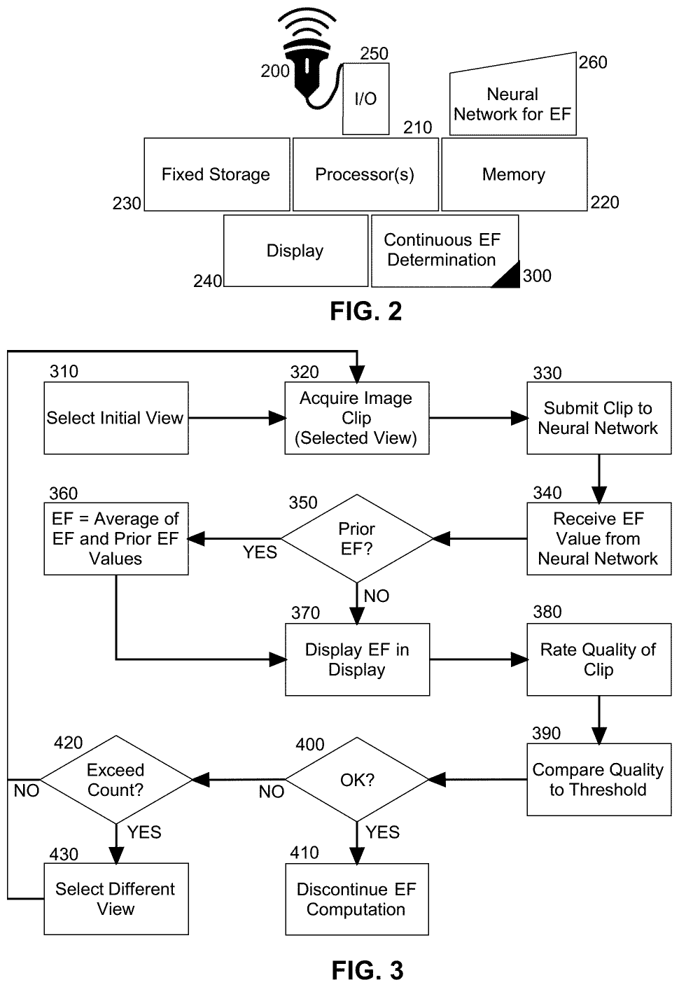

The process described in connection with FIG. 1 may be implemented within a computer data processing system. In further illustration, FIG. 2 schematically shows a data processing system adapted for the continuous and dynamic computation of ejection fraction. The system includes a host computing platform that includes one or more computers, each with at least one processor 210, memory 220, fixed storage 230 and a display 240. The fixed storage 230 stores therein ultrasound video clips of a target heart acquired by input/output circuitry 250 communicatively coupled to an ultrasound diagnostic imaging device 200.

A neural network 260 may be loaded at run time into the memory 220 of the host computing platform. The neural network 260 is trained to correlate different imagery of different video clips of different hearts with corresponding ejection fraction values so that when the neural network 260 is presented with a contemporaneously acquired video clip of the target heart, the neural network 260 returns a correlated ejection fraction value. Likewise, the neural network 260 is trained to correlate different video clips of different hearts using different views of the different hears with corresponding image quality values so that when the neural network 260 is presented with the contemporaneously acquired video clip of the target heart, the neural network 260 also returns a correlated image quality value.

Of note, the system yet further includes a continuous ejection fraction determination module 300. The continuous ejection fraction determination module 300 includes computer program instructions that when executing in the memory 220 by the one or more processors 210 of the host computing platform, loads into the memory 220 the neural network 260 and receives a continuous stream of different video clips, either previously acquired and stored in the fixed storage 230, or contemporaneously acquired in real time from the ultrasound diagnostic imaging device 200, and submits the video clips in succession to the neural network 260. Thereafter, the program instructions receive from the neural network in response, both an image quality value and also an ejection fraction value for a corresponding one of the video clips. The program instructions further present the image quality value and the ejection fraction value in the display 240 for each one of the video clips in the succession.

The program instructions repeat the display of the image quality value and the ejection fraction value in the display 240 until it is determined that the image quality value exceeds a threshold. But, prior to determining that the image quality value exceeds the threshold, after a specified lapse of time or after having received a succession of image quality values below the threshold, the view selected for acquiring the video clips can be changed, either automatically or manually in response to a prompt presented in the display 240 by the program instructions. The program instructions further can average a set of successive ejection fraction values and display only the computed average ejection fraction value. As well, the program instructions can average a set of successive video clips prior to submitting the video clips to the neural network 260 and, instead, the program instructions may submit the average of the video clips in the set to the neural network 260 in order to receive an ejection fraction value and an image quality value reflective of the average of the video clips.

In even further illustration of the operation of the continuous ejection fraction determination module 300, FIG. 3 is a flow chart illustrating a process for continuously and dynamically computing ejection fraction. Beginning in block 310 an initial view is selected for use in acquiring ultrasound imagery of a target heart. In block 320, a first video clip is received as acquired according to the selected view. In block 330, the video clip may be submitted to the neural network and, in response, in block 340 the neural network produces an ejection fraction value correlated with imagery in the video clip.

In decision block 350, it is determined if a prior ejection fraction value has been received from the neural network for a prior video clip acquired for the target heart. If not, the contemporaneous ejection fraction value is set to the produced ejection fraction value, but otherwise, in block 360, the contemporaneous ejection fraction value is computed as an average of the produced ejection fraction value and one or more of the prior ejection fraction values. Then, in block 370 the contemporaneous ejection fraction value is displayed in a user interface to the ultrasound diagnostic imaging device.

In block 380, a quality value for the quality of the video clip submitted to the neural network also may be received from the neural network and in block 390, the quality value is compared to a threshold value. In decision block 400, if it is determined that the quality value meets or exceeds the threshold value, then the process ends in block 410. But otherwise, in decision block 420 it is determined if the count of video clips acquired for the target heart using the selected view has exceeded a threshold count. If so, in block 430 a different view is selected for acquiring subsequent video clips and in block 320, the process repeats using the different view.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “include”, “includes”, and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for continuously and dynamically computing ejection fraction comprising:
training a neural network with different sets of cardiac imaging data acquired of a ventricle for different hearts and a known ejection fraction for each of the sets;
loading the trained neural network into memory of a computer;
continuously acquiring contemporaneous sets of imaging data of a ventricle of a heart according to a specified view;
for each corresponding one of the sets of imaging data, computing an image quality value for the corresponding one of the sets, providing the corresponding one of the sets of imaging data to the neural network and receiving as output from the neural network, an ejection fraction determination output without tracing a ventricle boundary of the heart, and displaying both the computed image quality value and the ejection fraction determination output in a display of the computer.

2. The method of claim 1, further comprising ending the continuous acquisition of the contemporaneous sets of imaging data responsive to a quality value that exceeds a threshold value.

3. The method of claim 1, further comprising:
determining that a threshold number of sets of imaging data have been acquired without computing a quality value that exceeds the threshold value;
selecting a different view than the specified view; and,
continuing the continuous acquisition of the contemporaneous sets of imaging data utilizing the different view.

4. The method of claim 1, further comprising:
selecting a different view than the specified view; and,
continuing the continuous acquisition of the contemporaneous sets of imaging data utilizing the different view.

5. The method of claim 1, wherein the continuous acquisition of the contemporaneous sets of imaging data comprise averaging at least two successive ones of the sets of imaging data and providing the averaged at least two successive ones of the sets of imaging data to the neural network.

6. The method of claim 1, further comprising averaging an ejection fraction determination for multiple different ones of the contemporaneous sets of imaging data to produce an averaged ejection fraction determination for display in the computer.

7. The method of claim 6, wherein each ejection fraction determination included in the averaging is weighted according to a corresponding computed image quality value.

8. The method of claim 1, further comprising:
selecting a different view than the specified view;
continuing the continuous acquisition of the contemporaneous sets of imaging data utilizing the different view; and,
averaging an ejection fraction determination for multiple different ones of the contemporaneous sets of imaging data to produce an averaged ejection fraction determination for display in the computer;
wherein each ejection fraction determination included in the averaging is weighted according to a corresponding image view.

9. A data processing system adapted for continuously and dynamically computing ejection fraction, the system comprising:
a host computing platform comprising one or more computers, each comprising memory and at least one processor;
a display communicatively coupled to the host computing platform;
a data store storing therein a neural network trained with different sets of cardiac imaging data acquired of a ventricle for different hearts and a known ejection fraction for each of the sets; and,
an ejection fraction determination module comprising computer program instructions enabled while executing in the host computing platform to perform:
loading the trained neural network into the memory of the computer;
continuously acquiring contemporaneous sets of imaging data of a ventricle of a heart according to a specified view;
for each corresponding one of the sets of imaging data, computing an image quality value for the corresponding one of the sets, providing the corresponding one of the sets of imaging data to the neural network and receiving as output from the neural network, an ejection fraction determination output without tracing a ventricle boundary of the heart, and displaying both the computed image quality value and the ejection fraction determination output in the display.

10. The system of claim 9, wherein the computer program instructions are further enabled to perform the ending the continuous acquisition of the contemporaneous sets of imaging data responsive to a quality value that exceeds a threshold value.

11. The system of claim 9, wherein the computer program instructions are further enabled to perform:
determining that a threshold number of sets of imaging data have been acquired without computing a quality value that exceeds the threshold value;
selecting a different view than the specified view; and,
continuing the continuous acquisition of the contemporaneous sets of imaging data utilizing the different view.

12. The system of claim 9, wherein the continuous acquisition of the contemporaneous sets of imaging data comprise averaging at least two successive ones of the sets of imaging data and providing the averaged at least two successive ones of the sets of imaging data to the neural network.

13. The system of claim 9, wherein the computer program instructions are further enabled to perform averaging an ejection fraction determination for multiple different ones of the contemporaneous sets of imaging data to produce an averaged ejection fraction determination for display in the computer.

14. A computer program product for continuously and dynamically computing ejection fraction, the computer program product including a computer readable storage medium having program instructions embodied therewith, the program instructions executable by a device to cause the device to perform a method including:
training a neural network with different sets of cardiac imaging data acquired of a ventricle for different hearts and a known ejection fraction for each of the sets;
loading the trained neural network into memory of a computer;
continuously acquiring contemporaneous sets of imaging data of a ventricle of a heart according to a specified view;
for each corresponding one of the sets of imaging data, computing an image quality value for the corresponding one of the sets, providing the corresponding one of the sets of imaging data to the neural network and receiving as output from the neural network, an ejection fraction determination output without tracing a ventricle boundary of the heart, and displaying both the computed image quality value and the ejection fraction determination output in a display of the computer.

15. The computer program product of claim 14, wherein the method further includes ending the continuous acquisition of the contemporaneous sets of imaging data responsive to a quality value that exceeds a threshold value.

16. The computer program product of claim 14, wherein the method further includes:
determining that a threshold number of sets of imaging data have been acquired without computing a quality value that exceeds the threshold value;
selecting a different view than the specified view; and,
continuing the continuous acquisition of the contemporaneous sets of imaging data utilizing the different view.

17. The computer program product of claim 14, wherein the continuous acquisition of the contemporaneous sets of imaging data comprise averaging at least two successive ones of the sets of imaging data and providing the averaged at least two successive ones of the sets of imaging data to the neural network.

18. The computer program product of claim 14, wherein the method further includes averaging an ejection fraction determination for multiple different ones of the contemporaneous sets of imaging data to produce an averaged ejection fraction determination for display in the computer.

19. The computer program product of claim 18, wherein each ejection fraction determination included in the averaging is weighted according to a corresponding computed image quality value.

20. The computer program product of claim 14, wherein the method further includes:
selecting a different view than the specified view;
continuing the continuous acquisition of the contemporaneous sets of imaging data utilizing the different view; and,
averaging an ejection fraction determination for multiple different ones of the contemporaneous sets of imaging data to produce an averaged ejection fraction determination for display in the computer;
wherein each ejection fraction determination included in the averaging is weighted according to a corresponding image view.
